(19) United States
(12) Patent Application Publication
(10) Pub. No.: US 2025/0358019 A1
(43) Pub. Date: Nov. 20, 2025

(54) SIGNAL DETECTION DEVICE

(71) Applicant: Electronics and Telecommunications Research Institute, Daejeon (KR)

(72) Inventors: Chanki PARK, Daejeon (KR); Hyun Soon SHIN, Daejeon (KR)

(73) Assignee: Electronics and Telecommunications Research Institute, Daejeon (KR)

(21) Appl. No.: 19/006,213

(22) Filed: Dec. 30, 2024

(30) Foreign Application Priority Data
May 14, 2024 (KR) 10-2024-0063217

(51) Int. Cl. H04B 13/00; H04B 1/08

(52) U.S. Cl. CPC H04B 13/005; H04B 1/08

(57) ABSTRACT

Disclosed is a signal detection device, which includes a first detection sensor set that detects input signals generated from a signal generator and reconstructs output signals based on the input signals, and the first detection sensor set includes a plurality of detection sensors, and at least some of the plurality of detection sensors are arranged in a sparse array having a sparser number of sensors than a minimum value of a density of a sensor array based on a Nyquist-Shannon sampling theorem.

### CROSS-REFERENCE TO RELATED APPLICATIONS

This application claims priority under 35 U.S.C. § 119 to Korean Patent Application No. 10-2024-0063217 filed on May 14, 2024, in the Korean Intellectual Property Office, the disclosures of which are incorporated by reference herein in their entireties.

### BACKGROUND

Embodiments of the present disclosure described herein relate to a signal detection device, and more particularly, relate to a brain activity detection device.

A brain is largely divided into a cerebrum and a cerebellum. The cerebrum is divided into detailed areas such as a frontal lobe, a temporal lobe, and an occipital lobe. The brain generates brain activity signals such as changes in blood volume in a cerebral cortex and brain waves. The cerebrum is composed of a cortex and a medulla, and the location of the cortex may be inferred through the brain activity signals. In addition, it is possible to infer each body function based on the location of the cortex.

The representative methods for measuring brain activity signals are an EEG (Electroencephalography) and a fNIRs (Functional Near-Infrared Spectroscopy). Compared to the fNIRs, the EEG has high temporal resolution but low spatial resolution. In contrast, the fNIRs has a shallow and narrow measurement range compared to the EEG. Recently, the development of a technology that combines the two methods is being discussed.

A Nyquist-Shannon sampling theorem, a widely known theory, indicates that lossless sampling is possible when the sampling rate is equal to or greater than twice the maximum frequency of an analog signal spectrum. In addition, in measuring spatial information of brain activity, the arrangement of sensors and the minimum value of the sensor array density are determined depending on the Nyquist-Shannon sampling theorem.

In arranging electrodes that detect brain activity signals, there is a need to accurately measure spatial information of brain activity with high accuracy while considering the convenience of a signal generating unit.

### SUMMARY

Embodiments of the present disclosure provide a device that optimizes the electrode arrangement to ensure high accuracy while considering the convenience of a signal acquisition unit when brain activity signals are detected.

According to an embodiment of the present disclosure, a signal detection device includes a first detection sensor set that detects input signals generated from a signal generator and reconstructs output signals based on the input signals, and the first detection sensor set includes a plurality of detection sensors, and at least some of the plurality of detection sensors are arranged in a sparse array having a sparser number of sensors than a minimum value of a density of a sensor array based on a Nyquist-Shannon sampling theorem.

According to an embodiment of the present disclosure, a signal detection method includes a first sensor arrangement operation of arranging a first detection sensor set including a plurality of detection sensors outside a signal generator, a signal detection operation of detecting, by the first detection sensor set, input signals generated from the signal generator, and an output operation of reconstructing, by the first detection sensor set, output signals based on the input signals, and the first sensor arrangement operation includes arranging at least some of the plurality of detection sensors in a sparse array having a sparser number of sensors than a minimum value of a density of a sensor array based on a Nyquist-Shannon sampling theorem.

### BRIEF DESCRIPTION OF THE FIGURES

The above and other objects and features of the present disclosure will become apparent by describing in detail embodiments thereof with reference to the accompanying drawings.

FIG. 1 is a diagram illustrating a brain-machine interface (BMI), according to an embodiment of the present disclosure.

FIG. 2 is a diagram illustrating an example of a linear sparse ruler array, as an arrangement method of a first detection sensor set.

FIG. 3 is a diagram illustrating an example of a circular sparse ruler array, as an arrangement method of a first detection sensor set.

FIG. 4 is a diagram illustrating a method of designing a symmetric linear sparse ruler array.

FIG. 5 is a diagram illustrating a case where a first detection sensor set of a symmetric linear sparse ruler array is arranged outside a brain, according to an embodiment of the present disclosure.

FIG. 6 is a diagram illustrating cases where a first detection sensor set arranged in a symmetric circular sparse ruler array is arranged outside a brain, according to an embodiment of the present disclosure.

FIG. 7 is a diagram illustrating a case where a first detection sensor set arranged in a symmetric spherical sparse ruler array is arranged outside a brain according to an embodiment of the present disclosure.

FIG. 8 is a diagram illustrating a case where a first detection sensor set and a second detection sensor set are arranged together, according to an embodiment of the present disclosure.

FIG. 9 is a diagram illustrating a brain activity detection method, according to an embodiment of the present disclosure.

### DETAILED DESCRIPTION

Hereinafter, embodiments of the present disclosure will be described in detail and clearly to such an extent that an ordinary one in the art easily implements the present disclosure.

The present disclosure is not limited to the embodiments disclosed below, but may be implemented in various forms and various modifications and changes may be applied. However, it is provided to complete the disclosure of the present disclosure through the description of the present embodiment, and to completely inform those skilled in the art of the scope of the disclosure to which the present disclosure belongs. In the accompanying drawings, for convenience of description, the size of the components is illustrated larger than the actual size, and the ratio of each component may be exaggerated or reduced.

Although terms such as first, second, and third are used to describe various components in various embodiments of the present specification, these components should not be limited by these terms. These terms are only used to distinguish one component from another component. Embodiments described and illustrated herein also include complementary embodiments thereof.

The terms used herein are provided to describe the embodiments but not to limit the present disclosure. In addition, unless otherwise defined, terms used in the embodiments of the present disclosure may be interpreted as meanings commonly known to those skilled in the art.

In the specification, the singular forms include plural forms unless particularly mentioned. As used herein, “comprises” and/or “comprising” does not exclude the presence or addition of one or more other components, steps, operations and/or elements to the mentioned components, steps, operations and/or elements.

Reference throughout this specification to “one embodiment” or “an embodiment” means that a particular feature, structure, or characteristic described in connection with embodiments may be included in at least one embodiment disclosed herein. Thus, appearances of the phrases (or other phrases having a similar meaning) “in one embodiment” or “in an embodiment” or “according to an embodiment” in various places throughout this specification are not necessarily all referring to the same embodiment. Also, particular features, structures, or characteristics may be combined in any suitable way in one or more embodiments. In this regard, as used herein, the word “exemplary” means “providing an example, instance, or illustration.” Any embodiment described herein as “exemplary” should not necessarily be construed as preferred or advantageous over other embodiments.

Unless defined otherwise, all terms (including technical and scientific terms) used herein have the same meaning as commonly understood by one of ordinary skill in the art to which this disclosure belongs.

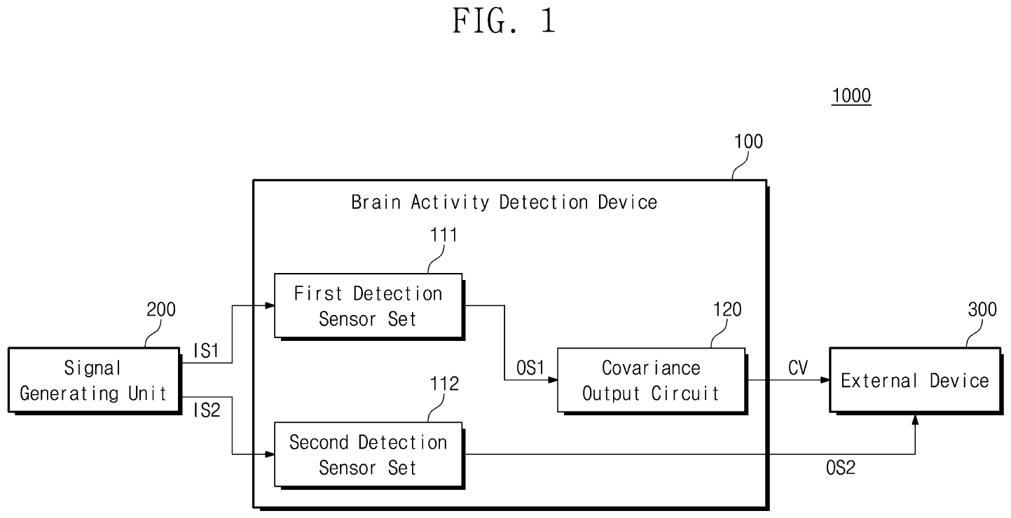

FIG. 1 is a diagram illustrating a brain-machine interface (BMI) 1000, according to an embodiment of the present disclosure.

Referring to FIG. 1, the brain-machine interface 1000 may be connected to an external device 300. For example, the external device 300 may include a machine, a computer, etc. The brain-machine interface 1000 may be configured to output characteristics or properties of a signal to the external device 300, based on the signal generated from a signal generating unit 200. For example, the brain-machine interface 1000 may be configured to measure brain activity signals generated from a brain, to extract the characteristics or properties of the acquired brain activity signals, and to output the extracted characteristics or properties to the external device 300.

The brain-machine interface 1000 may include a brain activity detection device 100 and the signal generating unit 200.

The brain activity detection device 100 may be attached to the signal generating unit 200 and may be configured to detect a first input signal IS1 and a second input signal IS2. For example, the signal generating unit 200 may be configured to output the first input signal IS1 and the second input signal IS2. For example, the signal generating unit 200 may represent the brain of a person using the brain activity detection device 100. In this case, the first input signal IS1 and the second input signal IS2 may represent brain activity signals. For example, the brain activity detection device 100 may be configured to be non-invasively attached to the brain and to detect brain activity. Hereinafter, the present specification will be described through an example in which the signal generating unit 200 is the brain of a person using the brain activity detection device 100, but the present disclosure is not necessarily limited thereto.

The brain activity detection device 100 may include a first detection sensor set 111, a second detection sensor set 112, and a covariance output circuit 120.

The first detection sensor set 111 and the second detection sensor set 112 may be configured to detect the first input signal IS1 and the second input signal IS2. For example, the first input signal IS1 may include brain waves, which are potentials generated by electrical activity of neurons in the brain. For example, the second input signal IS2 may include changes in blood volume of the cerebral cortex induced by activity of neurons in the brain.

The first detection sensor set 111 and the second detection sensor set 112 may include a plurality of detection sensors. According to one embodiment, the first detection sensor set 111 and the second detection sensor set 112 may include a plurality of detection sensors configured to use methods such as an EEG (Electroencephalography), an fNIRs (Functional Near-Infrared Spectroscopy), an fMRI (Functional Magnetic Resonance Imaging), an EMG (Electromyography), and/or an fEMG (Functional Electromyography). For example, the first detection sensor set 111 may include brain wave detection electrodes configured to detect brain wave signals. For example, the first detection sensor set 111 may be EEG electrodes. For example, the second detection sensor set 112 may include a signal sensor configured to detect changes in blood volume of the cerebral cortex. For example, the second detection sensor set 112 may include an fNIRs transmitter and an fNIRs receiver.

The first detection sensor set 111 and the second detection sensor set 112 may be configured to detect the first input signal IS1 and the second input signal IS2 in different areas of the signal generating unit 200, respectively. For example, the local areas where the first detection sensor set 111 and the second detection sensor set 112 are attached to the signal generating unit 200 may be different from each other. For example, the first input signal IS1 received by the first detection sensor set 111 may represent brain wave signals of a local area to which the first detection sensor set 111 is attached. For example, the second input signal IS2 received by the second detection sensor set 112 may indicate a change in blood volume of the cerebral cortex of a local area to which the second detection sensor set 112 is attached. For example, the brain activity detection device 100 may measure brain wave information for the entire brain based on the first input signal IS1 received by the first detection sensor set 111.

According to the comparative example of the present disclosure, the first detection sensor set 111 may be arranged according to a 10-20 system. The 10-20 system may measure brain wave information by arranging EEG electrodes in specific areas such as the frontal lobe, the temporal lobe, and the occipital lobe of the brain. The 10-20 system may arrange electrodes based on the distance between the front and the back of a skull of the brain or the distance between two sides of the skull. For example, the 10-20 system may place the electrodes at intervals of 10% and/or 20% of the distance between the front and the back of the skull of the brain. For example, the 10-20 system may place the electrodes at intervals of 10% and/or 20% of the distance between the both sides of the skull of the brain.

In the 10-20 system, the minimum value of the density of the sensor arrangement may be determined based on the Nyquist-Shannon sampling theorem. For example, based on the Nyquist-Shannon sampling theorem, the minimum value of the density of the sensor arrangement may be calculated through the maximum frequency of the brain wave signal to be measured. For example, the maximum frequency of the brain wave signal to be measured may be 100 Hz. For example, the maximum interval of the sensor arrangement may be calculated through Equation 1. For example, the minimum value of the density of the sensor arrangement may be determined based on the maximum interval between the detection sensors and the area of the measurement area.

$${d}_{H} = \frac{s}{2 * f} \qquad [ Equation 1 ]$$

Based on the Nyquist-Shannon sampling theorem, d̅_̅{̅H̅}̅ is a maximum interval between detection sensors, f̅ is a maximum frequency of the brain wave signal to be measured, and the “s” represents a propagation velocity of the brain wave signal.

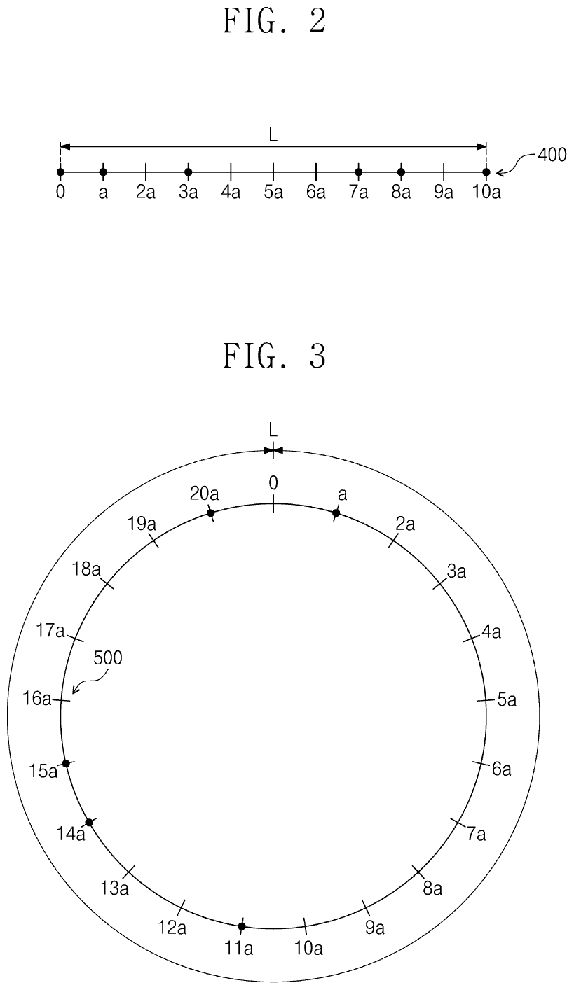

According to an embodiment of the present disclosure, the first detection sensor set 111 may be arranged in a sparse arrangement that is sparser than the minimum value of the density of the sensor arrangement required according to the Nyquist-Shannon sampling theorem. A specific description of the sparse arrangement of the present disclosure will be described later with reference to FIGS. 2 to 7.

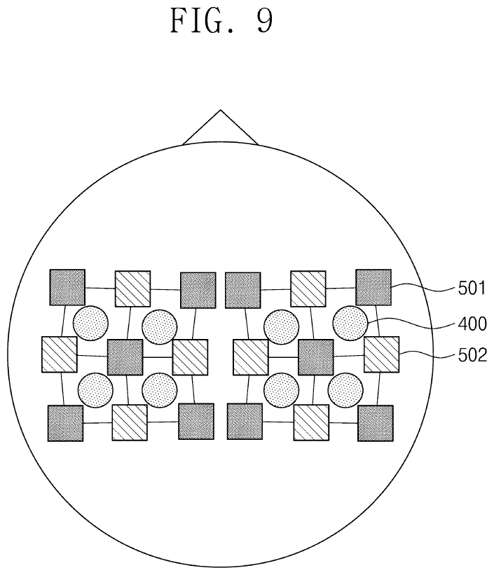

When the first detection sensor set 111 is arranged in the sparse arrangement, the second detection sensor set 112 may be arranged in a free space outside the signal generating unit 200. For example, the second detection sensor set 112 may be arranged between the sparse arrangements of the first detection sensor set 111. A specific description of the manner in which the second detection sensor set 112 is arranged will be described later with reference to FIG. 8.

The first detection sensor set 111 and the second detection sensor set 112 may be configured to output a first output signal OS1 and a second output signal OS2 based on the first input signal IS1 and the second input signal IS2. For example, the first output signal OS1 may represent the brain wave signal detected from the brain. For example, the second output signal OS2 may represent a change in blood volume of the cerebral cortex detected from the brain. For example, the first detection sensor set 111 may be configured to provide the first output signal OS1 to the covariance output circuit 120, and the second detection sensor set 112 may be configured to provide the second output signal OS2 to the external device 300.

The covariance output circuit 120 may be configured to output covariance vectors or matrices CV of the first output signal OS1. For example, the covariance output circuit 120 may be configured to apply various algorithms or operations to the first output signal OS1 to output the covariance vectors or matrices CV of the first output signal OS1.

The external device 300 may be configured to output features of brain activity signals through the covariance vectors or matrices CV. For example, the external device 300 may be configured to output features of brain activity signals based on training through an artificial neural network using the covariance vectors or matrices CV. For example, the features of brain activity signals may represent changes in voltage, current, amplitude, magnitude, frequency, phase, etc. over time. For example, the features of brain activity signals may represent areas where neurons in the brain are activated.

With the above configuration, the brain activity detection device according to the embodiments of the present disclosure may detect brain activity using detection sensors arranged in the sparse array. Therefore, the measurement cost may be reduced when the brain activity is detected, and convenience may be increased when worn over the signal generating unit. In addition, a sparse array that is symmetrical on the left and right may be designed, so that simple design of a sparse array is possible. By sparsely arranging the detection sensors, detection sensors with different measurement methods and measurement ranges may be attached to the created free space, thereby enabling the brain activity to be detected with higher accuracy.

FIG. 2 is a drawing illustrating an example of a linear sparse ruler array, as an arrangement method of the first detection sensor set 111.

The first detection sensor set 111 may be arranged linearly (refer to FIG. 1). For example, the first detection sensor set 111 may be arranged in a straight line or a curve. As an example, the first detection sensor set 111 may be arranged in a linear sparse ruler array.

Referring to FIG. 2, a first arrangement line 400 including a plurality of mark points may be defined. A mark point may mean an area where detection sensors may be provided on the first arrangement line 400. For example, the first arrangement line 400 may be a straight line or a curve. The intervals of the plurality of mark points may mean a moving distance along a straight line or a curve. Mark points may always be arranged at both ends of the first arrangement line 400. For example, the plurality of mark points may include a first mark point to an x-th mark point. The first detection sensor set 111 may be arranged on at least some of the plurality of mark points on the first arrangement line 400.

The first detection sensor set 111 may be arranged in a×K-set pattern so as to satisfy a set condition of Equation 2. When the first detection sensor set 111 is arranged so as to satisfy the set condition of Equation 2, it represents the case where the first detection sensor set 111 is arranged in a linear sparse ruler array.

$$K ⊂ \{ 0 , … , N - 1 \} for all integer l between 0 and N - 1 , \\ there exists at least one pair of elements k and {k}^{′} in K \\ such that k - {k}^{′} = l \qquad [ Equation 2 ]$$

Referring to FIG. 2, as an example, mark points may be arranged in 0, a, 2a, . . . , L areas on the first arrangement line 400 having a length of “L” through Equations 2. For example, the first detection sensor set 111 may be arranged in 0, a, 3a, 7a, 8a, 10a areas, based on Equation 2. However, when the first detection sensor set 111 is arranged based on Equation 2, there may be various cases, different from those illustrated in FIG. 2.

The first detection sensor set 111 does not necessarily need to be arranged in a linear sparse ruler array, and unlike FIG. 2, the sparse array method of the plurality of detection sensors may be arranged in a nested array method, a co-prime array method, etc.

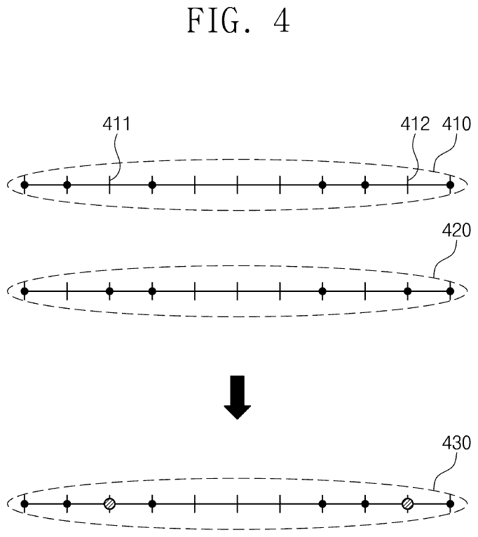

FIG. 3 is a diagram illustrating an example of a circular sparse ruler array as an arrangement manner of the first detection sensor set 111.

The first detection sensor set 111 may be arranged in a circular shape (refer to FIG. 1). As an example, the first detection sensor set 111 may be arranged in a circular sparse ruler array.

Referring to FIG. 3, a second arrangement line 500 including a plurality of mark points may be defined. A mark point may mean an area where detection sensors may be provided on the second arrangement line 500. The second arrangement line 500 may be circular. The interval of the plurality of mark points may mean a moving distance along a curve. For example, the plurality of mark points may include a first mark point to an x-th mark point. The first detection sensor set 111 may be arranged on at least some of the plurality of mark points on the second arrangement line 500.

The interval between the mark points may be less than the maximum interval between the detection sensors.

The first detection sensor set 111 may be arranged in a×K-set pattern so as to satisfy a set condition of Equation 3. In the case where the first detection sensor set 111 is arranged so as to satisfy the set condition of Equation 3, it represents a case where the first detection sensor set 111 is arranged in a circular sparse ruler array.

$$K ⊂ \{ 0 , … , N - 1 \} for all integer l between 0 and N - 1 , \\ there exists at least one or more pairs k and {k}^{′} in K for \\ any integer l from 0 to N - 1 such that mod ( k - {k}^{′} , N ) = l \qquad [ Equation 3 ]$$

Where, the “modo” represents the modulo operator.

Referring to FIG. 3, as an example, mark points may be arranged in 0, a, 2a, . . . , L areas on the second arrangement line 500 of a length of “L”. For example, the first detection sensor set 111 may be arranged in 0, a, 11a, 14a, 15a, 20a areas, based on Equation 3. However, when the first detection sensor set 111 is arranged based on Equation 3, there may be various cases, different from those illustrated in FIG. 3.

The first detection sensor set 111 does not necessarily need to be arranged in a circular sparse ruler array, and unlike FIG. 3, the sparse array method of the first detection sensor set 111 may be arranged in a nested array method, a co-prime array method, etc.

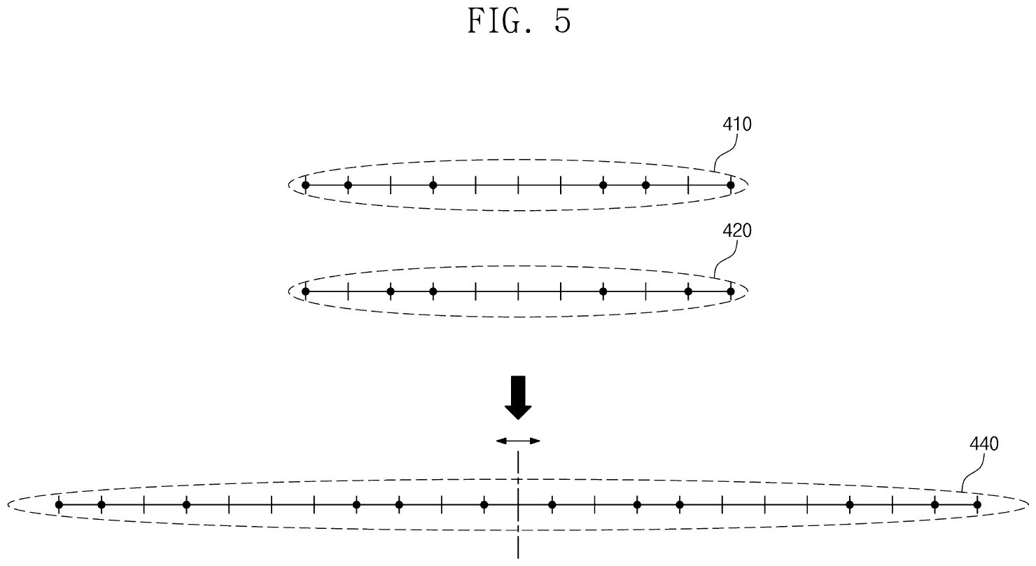

FIG. 4 is a diagram illustrating a method of designing a symmetric linear sparse ruler array.

The first detection sensor set 111 may be arranged symmetrically left and right with respect to a center of the brain. The first detection sensor set 111 may be placed on the brain as a symmetrical sparse ruler array, which is symmetrical left and right with respect to the center of the brain. Depending on the left-right symmetry of the brain, the sparse array may be designed symmetrically, allowing for a simple design of the sparse array.

As an example, the symmetrical linear sparse ruler array may be designed by symmetrizing a linear sparse ruler array left and right.

Referring to FIG. 4, a first linear sparse ruler array 410 may be asymmetrically left and right with respect to the center. A second linear sparse ruler array 420 may be designed by symmetrizing the first linear sparse ruler array 410 left and right. When the first linear sparse ruler array 410 and the second linear sparse ruler array 420 are overlapped, the detection sensors of a first mark point 411 and a second mark point 412 may not overlap. A third linear sparse array 430 may be designed by placing detection sensors at the first mark point 411 and the second mark point 412 of the first linear sparse ruler array 410.

Although not illustrated, a symmetrical circular sparse ruler array may also be designed in the same manner as FIG. 4.

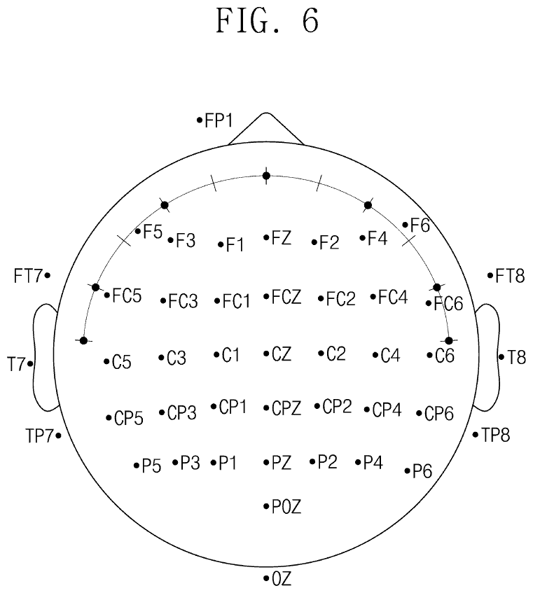

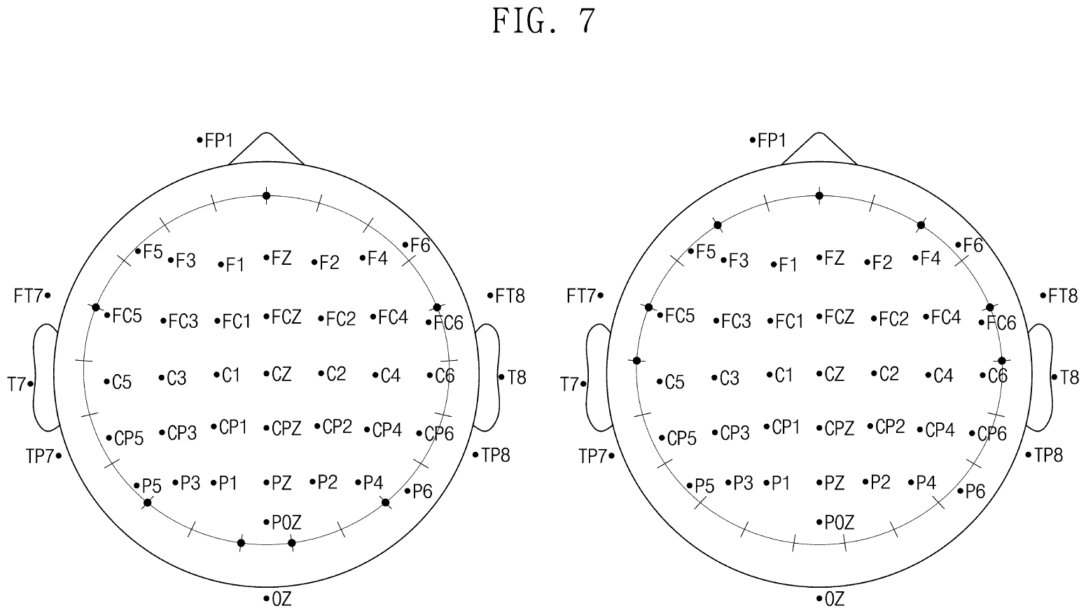

FIG. 5 is a diagram illustrating a case where the first detection sensor set 111 of a symmetrical linear sparse ruler array is arranged outside a brain, according to an embodiment of the present disclosure. FIG. 6 is a diagram illustrating cases where the first detection sensor set 111 arranged in a symmetrical circular sparse ruler array is arranged outside the brain, according to an embodiment of the present disclosure. Hereinafter, the description will be given with reference to FIG. 5 and FIG. 6 together.

Referring to FIGS. 5 and 6, for example, “F” represents a frontal lobe, “T” represents a temporal lobe, TP represents a temporo-parietal lobe, “P” represents a parietal lobe, PO represents the parieto-occipital lobe, and “O” represents an occipital lobe.

The linear sparse ruler array and the circular sparse ruler array may always have an area where detection sensors are densely placed.

As an example, in the case of the linear sparse ruler array, the first detection sensor set 111 may be arranged on a forehead outside the brain. In the case of the circular sparse ruler array, the first detection sensor set 111 may be arranged around the head outside the brain. For example, the area where the first detection sensor set 111 is densely placed may be arranged in an area where the brain activity detection device 100 outside the brain may be easily attached. For example, when the first detection sensor set 111 is arranged in a circular sparse ruler array, the area where the detection sensors are densely placed may be arranged on the forehead.

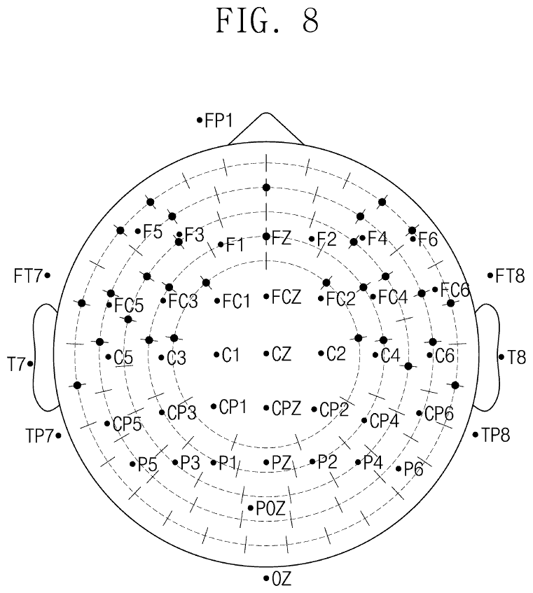

FIG. 7 is a diagram illustrating a case where the first detection sensor set 111 arranged in a symmetrical spherical sparse ruler array is arranged outside the brain, according to an embodiment of the present disclosure.

The spherical sparse ruler array may always have an area where the detection sensors are densely placed.

As an example, the first detection sensor set 111 of the symmetric spherical sparse ruler array may be arranged over an entire scalp outside the brain. The area where the first detection sensor set 111 of the symmetric spherical sparse ruler array is densely placed may be arranged in an area where the brain activity detection device 100 outside the brain may be easily attached.

The symmetric spherical sparse ruler array may be designed through a combination of a plurality of symmetrical linear sparse ruler arrays and a plurality of symmetrical circular sparse ruler arrays.

As an example, a symmetric spherical sparse ruler array may be designed by combining the first symmetric circular sparse ruler array to an n-th symmetric circular sparse ruler array. For example, a length of an m-th symmetric circular sparse ruler array may be less than a length of an (m+1)-th symmetric circular sparse ruler array (where, “m” is an integer 1≤m≤n−1). A length of a first symmetric circular sparse ruler array may be less than a length of a second symmetric circular sparse ruler array. For example, the m-th symmetric circular sparse ruler array may be arranged inside the circle of the (m+1)-th symmetric circular sparse ruler array (where, “m” is an integer 1≤m≤n−1). The first symmetric circular sparse ruler array may be arranged inside the circle of the second symmetric circular sparse ruler array.

For example, referring to FIG. 7, the symmetric spherical sparse ruler array may be designed through a combination of the first symmetric circular sparse ruler array to a fifth symmetric circular sparse ruler array.

FIG. 8 is a diagram illustrating a case where the first detection sensor set 111 and the second detection sensor set 112 are arranged together, according to an embodiment of the present disclosure.

The first detection sensor set 111 and the second detection sensor set 112 may be arranged in combination outside the brain. The first detection sensor set 111 may be arranged outside the brain according to the sparse array of the present disclosure. For example, when the first detection sensor set 111 is arranged according to the sparse array, the second detection sensor set 112 may be arranged in a free space outside the brain. For example, the second detection sensor set 112 may be arranged between the first detection sensor sets 111. For example, the second detection sensor set 112 may be arranged in a free space outside the brain, according to the sparse arrangement of the present disclosure.

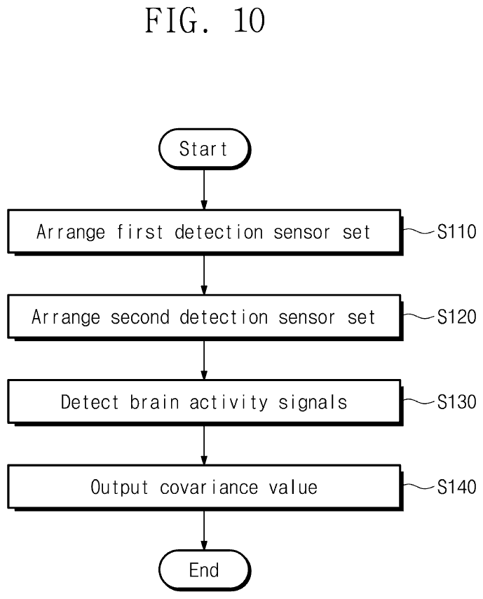

FIG. 9 is a diagram illustrating a brain activity detection method, according to an embodiment of the present disclosure. Hereinafter, with reference to FIG. 1, a method for detecting brain activity will be described.

Referring to FIG. 9, in operation S110, the first detection sensor set 111 may be arranged in a sparse array outside the signal generating unit 200. For example, the first detection sensor set 111 may be arranged in a sparse array outside the brain. For example, the method for arranging the first detection sensor set 111 in a sparse array may be as described in FIGS. 2 to 7.

In operation S120, the second detection sensor set 112 may be arranged. For example, the method for arranging the second detection sensor set 112 may be as described in FIG. 8.

In operation S130, the first detection sensor set 111 and the second detection sensor set may detect brain activity signals. The method by which the first detection sensor set 111 and the second detection sensor set detect brain activity signals may be as described in FIG. 1.

In operation S140, the covariance output circuit 120 may output covariance vectors or matrices based on the brain activity signal. The method by which the covariance output circuit 120 outputs the covariance vectors or matrices may be as described in FIG. 1.

As described above, the brain activity detection device according to the embodiments of the present disclosure may detect brain activity using detection sensors arranged in a sparse array. Therefore, the measurement cost may be reduced when the brain activity is detected, and convenience may be increased when worn over the signal generating unit. In addition, a sparse array that is symmetrical on the left and right may be designed, so that simple design of a sparse array is possible. By sparsely arranging the detection sensors, detection sensors with different measurement methods and measurement ranges may be attached to the created free space, thereby enabling the brain activity to be detected with higher accuracy.

As used herein, the terms “device” or “unit” refer to any combination of software, firmware, and/or hardware configured to provide the functionality described herein. For example, software may be implemented as a software package, code and/or set of instructions or instructions, and hardware, for example, may include hardwired circuitry, programmable circuitry, state machine circuitry, and/or a single or any combination, or assembly of firmware that stores instructions executed by programmable circuitry.

According to an embodiment of the present disclosure, the device having an optimized sensor arrangement that has high accuracy while considering the convenience of the signal generating unit when detecting brain activity signals is provided.

According to an embodiment of the present disclosure, the brain activity detection device may detect brain activity using the detection sensors arranged in a sparse array. Therefore, the measurement cost may be reduced when the brain activity is detected, and convenience may be increased when worn over the signal generating unit. In addition, a sparse array that is symmetrical on the left and right may be designed, so that simple design of a sparse array is possible. By sparsely arranging the detection sensors, detection sensors with different measurement methods and measurement ranges may be attached to the created free space, thereby enabling the brain activity to be detected with higher accuracy.

The above description refers to embodiments for implementing the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A signal detection device comprising:
a first detection sensor set configured to detect input signals generated from a signal generator and to reconstruct output signals based on the input signals, and
wherein the first detection sensor set includes a plurality of detection sensors, and
wherein at least some of the plurality of detection sensors are configured to be arranged in a sparse array having a sparser number of sensors than a minimum value of a density of a sensor array based on a Nyquist-Shannon sampling theorem.

2. The signal detection device of claim 1, wherein the signal generator represents a brain, and the input signals represent brain activity signals.

3. The signal detection device of claim 2, wherein the first detection sensor set uses an EEG (electroencephalography) method, and the plurality of detection sensors are configured with EEG electrodes so as to be arranged in the sparse array.

4. The signal detection device of claim 1, further comprising:
a covariance output circuit configured to generate covariance vectors or matrices of the input signals.

5. The signal detection device of claim 2, wherein the sparse array is arranged in a linear sparse ruler array, a circular sparse ruler array, a spherical sparse ruler array, a nested array, or a co-prime array.

6. The signal detection device of claim 5, wherein the linear sparse ruler array or the circular sparse ruler array is arranged on an arrangement line, and
wherein the arrangement line includes a plurality of mark points which are areas where a provision of the detection sensors is possible.

7. The signal detection device of claim 6, wherein the linear sparse ruler array is configured to be arranged in a×K-set pattern so as to satisfy a set condition of Equation 1,
$$K ⊂ \{ 0 , … , N - 1 \} for all integer l between 0 and N - 1 , \\ there exists at least one pair of elements k and {k}^{′} in K \\ such that k - {k}^{′} = l \qquad [ Equation 1 ]$$
wherein, the “a” represents a minimum interval between the mark points,
wherein, “a” is equal to “d_{H}” whose maximum value is determined by the Nyquist-Shannon sampling theorem. As a result, the detection sensors are arranged in the pattern of a×K and
the total length of the sensor “L” is equal to (N−1)×a.

8. The signal detection device of claim 6, wherein the circular sparse ruler array is configured to be arranged in a×K-set pattern so as to satisfy a set condition of Equation 2,
$$K ⊂ \{ 0 , … , N - 1 \} for all integer l between 0 and N - 1 , \\ there exists at least one or more pairs k and {k}^{′} in K for any integer l from 0 to N - 1 such that mod ( k - {k}^{′} , N ) = l \qquad [ Equation 2 ]$$
wherein, “a” means a minimum interval between the detection sensors and is equal to “d_{H}” whose maximum value is determined by the Nyquist-Shannon sampling theorem. As a result, the detection sensors are arranged in the pattern of a×K and
the total length of the sensor “L” is equal to N×a.

9. The signal detection device of claim 4, wherein an external device configured to output an area in which neurons of the brain are activated based on the covariance vectors or matrices, or
to output changes in voltage, current, amplitude, magnitude, frequency, phase, etc. of the brain activity signals over time.

10. The signal detection device of claim 5, wherein the linear sparse ruler array, the circular sparse ruler array, or the spherical sparse ruler array is configured to be symmetrical left and right around the brain.

11. The signal detection device of claim 7, wherein the spherical sparse ruler array is composed of a combination of a first circular sparse ruler array to an n-th circular sparse ruler array, and
in the first circular sparse ruler array to the n-th circular sparse ruler array:
wherein a length of an m-th circular sparse ruler array is shorter than a length of an (m+1)-th circular sparse ruler array, and
wherein the m-th circular sparse ruler array is arranged inside the (m+1)-th circular sparse ruler array, (where, “n” means an integer greater than or equal to “2”, and “m” means an integer of 1≤m≤n−1).

12. The signal detection device of claim 5, wherein the linear sparse ruler array is placed on a forehead outside the brain, and
wherein the circular sparse ruler array is placed around a head outside the brain.

13. The signal detection device of claim 3, wherein a second detection sensor set, and
wherein the second detection sensor set is arranged in a free space outside the brain, other than an area where the first detection sensor set is arranged, and
wherein the second detection sensor set is configured to measure the brain activity signals in a different way from the first detection sensor set.

14. A signal detection method comprising:
a first sensor arrangement operation of arranging a first detection sensor set including a plurality of detection sensors outside a signal generator;
a signal detection operation of detecting, by the first detection sensor set, input signals generated from the signal generator; and
an output operation of reconstructing, by the first detection sensor set, output signals based on the input signals, and
wherein the first sensor arrangement operation includes:
arranging at least some of the plurality of detection sensors in a sparse array having a sparser number of sensors than a minimum value of a density of a sensor array based on a Nyquist-Shannon sampling theorem.

15. The signal detection method of claim 14, wherein the signal generator represents a brain, and the input signals represent brain activity signals.

16. The signal detection method of claim 15, wherein the sparse array is arranged in a linear sparse ruler array, a circular sparse ruler array, a spherical sparse ruler array, a nested array, or a co-prime array.

17. The signal detection method of claim 16, wherein the linear sparse ruler array or the circular sparse ruler array is arranged on an arrangement line, and
wherein the arrangement line includes a plurality of mark points which are areas where a provision of the detection sensors is possible.

18. The signal detection method of claim 17, wherein the linear sparse ruler array is configured to be arranged in a×K-set pattern so as to satisfy a set condition of Equation 3,
$$K ⊂ \{ 0 , … , N - 1 \} for all integer l between 0 and N - 1 , \\ there exists at least one pair of elements k and {k}^{′} in K \\ such that k - {k}^{′} = l \qquad [ Equation 3 ]$$
wherein, the “a” represents a minimum interval between the mark points,
wherein, “a” is equal to “d_{H}” whose maximum value is determined by the Nyquist-Shannon sampling theorem. As a result, the detection sensors are arranged in the pattern of a×K and
the total length of the sensor “L” is equal to (N−1)×a.

19. The signal detection method of claim 17, wherein the circular sparse ruler array is configured to be arranged in a×K-set pattern so as to satisfy a set condition of Equation 4,
$$K ⊂ \{ 0 , … , N - 1 \} for all integer l between 0 and N - 1 , \\ there exists at least one or more pairs k and {k}^{′} in K for \\ any integer l from 0 to N - 1 such that mod ( k - {k}^{′} , N ) = l \qquad [ Equation 4 ]$$
wherein, “a” means a minimum interval between the detection sensors and is equal to “d_{H}” whose maximum value is determined by the Nyquist-Shannon sampling theorem. As a result, the detection sensors are arranged in the pattern of a×K and
the total length of the sensor “L” is equal to N×a.

20. The signal detection method of claim 16, wherein the linear sparse ruler array, the circular sparse ruler array, or the spherical sparse ruler array is configured to be symmetrical left and right around the brain.

21. The signal detection method of claim 16, wherein the spherical sparse ruler array is composed of a combination of a first circular sparse ruler array to an n-th circular sparse ruler array, and
in the first circular sparse ruler array to the n-th circular sparse ruler array:
wherein a length of an m-th circular sparse ruler array is shorter than a length of an (m+1)-th circular sparse ruler array, and
wherein the m-th circular sparse ruler array is arranged inside the (m+1)-th circular sparse ruler array, (where, “n” means an integer greater than or equal to “2”, and “m” means an integer of 1≤m≤n−1).

22. The signal detection method of claim 15, further comprising:
a second sensor arrangement operation of arranging a second detection sensor set including a plurality of detection sensors outside a signal generator, between the first sensor arrangement operation and the signal detection operation, and
wherein the second detection sensor set is arranged in a free space outside the brain, other than an area where the first detection sensor set is arranged, and
wherein the second detection sensor set is configured to measure the brain activity signals in a different way from the first detection sensor set.
